(19) United States
(12) Patent Application Publication
(10) Pub. No.: US 2026/0325552 A1
(43) Pub. Date: Oct. 22, 2026

(54) TOPICAL SPIRONOLACTONE AND MINOXIDIL COMPOSITIONS

(71) Applicant: Yogesh DANDIKER, Edina, MN (US)

(72) Inventor: Yogesh DANDIKER, Edina, MN (US)

(21) Appl. No.: 19/649,474

(22) Filed: Apr. 16, 2026

Related U.S. Application Data
(60) Provisional application No. 63/789,813, filed on Apr. 16, 2025.

(51) Int. Cl. A61K 31/585; A61K 9/00; A61K 9/12; A61K 31/455; A61K 31/506; A61K 47/10; A61K 47/12; A61K 47/14; A61K 47/22

(52) U.S. Cl. CPC A61K 31/585; A61K 9/0014; A61K 9/124; A61K 31/455; A61K 31/506; A61K 47/10; A61K 47/12; A61K 47/14; A61K 47/22

(57) ABSTRACT

The present disclosure provides a liquid composition free of C₁-C₃ alcohols and propylene glycol, comprising about 1% w/v to about 10% w/v spironolactone (and/or canrenone) and minoxidil, about 10% w/v to about 60% w/v C₄-C₁₄ aliphatic solvent, penetration enhancers, and including emollients, humectants and a conditioner wherein the composition is non-irritant when applied to the scalp. The disclosure also provides a method of treating male and female pattern hair loss in a subject in need thereof, the method comprising topically applying the composition described herein as a solution, gel, cream, foam or spray to the scalp of the subject.

### FIELD OF THE INVENTION

The present disclosure provides a liquid composition comprising about 1% w/v to about 20% w/v spironolactone and/or canrenone, about 1% w/v to about 20% w/v minoxidil, about 10% w/v to about 60% w/v of aliphatic solvents (e.g., a C₄-C₁₄ aliphatic solvents), a penetration enhancer, and about 1% w/v to about 30% w/v polyethylene glycol, wherein the composition does not comprise C₁-C₃ alcohols or propylene glycol. The disclosure further provides a method of treating hair loss in a human, the method comprising applying the composition to the scalp.

### BACKGROUND

Androgenetic alopecia (AGA) is a common form of hair loss in both men and women that affects 80% of men and 50% of women by the age of seventy. AGA consists of a characteristic receding frontal hairline in men and diffuse hair thinning in women with frontal hairline retention. The condition is primarily mediated by 5-alpha-reductase and dihydrotestosterone (DHT) which causes hair follicles to undergo miniaturization and shortening of successive anagen cycles. The extent and intensity of hair loss varies greatly between frontotemporal recession and residual occipital band, and patients may go through several stages in between.

Topical minoxidil, initially developed as a potent peripheral vasodilator agent for the treatment of severe refractory hypertension, is the mainstay treatment approved by the U.S. Food and Drug Administration (FDA) for androgenetic alopecia and is used as an off-label treatment for other hair loss conditions including alopecia areata and scarring alopecia. The mechanism by which minoxidil promotes hair growth is not fully understood. Minoxidil is an adenosine 5′-triphosphate-sensitive potassium channel opener causing hyperpolarization of cell membranes. Theoretically, by widening blood vessels and opening potassium channels, it allows more oxygen, blood, and nutrients to the follicles. Moreover, minoxidil contains a nitric oxide moiety and may act as a nitric oxide agonist causing follicles in the telogen phase to shed, which are then replaced by thicker hairs in a new anagen phase. In evaluating minoxidil's effectiveness in stimulating hair regrowth in men, investigators found that a topical 5% minoxidil solution was very effective in 15.9% of patients, effective in 47.8%, moderately effective in 20.6%, and ineffective. 15.7%.

Currently, topical minoxidil for treatment of AGA is available as a 2% solution or a 5% foam. However, these marketed minoxidil formulations have several issues. Firstly, in order to be effective, minoxidil needs to be converted by the enzyme sulfotransferase into its active form, minoxidil sulfate. Studies indicate that approximately 30-40% of women do not have sufficient levels of sulfotransferase in their hair follicles, which may lead to a reduced response to minoxidil treatment in women specifically and may explain why a significant portion of women do not experience the same effects from using minoxidil for hair loss as men do.

Secondly, minoxidil is poorly soluble in solution and concentrations greater than 5% tend to be unstable and crystallization (or powdering) of minoxidil occurs on the scalp when the solvent evaporates. The 5% foam formulation leaves a residue in hair which makes hair unmanageable and therefore is not preferred by many women. Solution formulations comprising minoxidil often contain propylene glycol to solubilize minoxidil. Evidence suggests that patients treated with alcohol-based minoxidil topical solutions that contain propylene glycol, e.g., compositions comprising C₁-C₃ alcohols, have been reported to cause local irritation of the scalp, pruritus, itching, burning, dryness, and redness and 11% of patients reported allergic contact dermatitis after using alcohol-based minoxidil solutions. Further, topical application of minoxidil results in variable systemic absorption depending on various factors, including the vehicle in the formulation, the area of application and skin status (absorption is increased if the scalp is abraded or inflamed). On average, only 1.4% of the active ingredient is systemically absorbed after topical application to the scalp.

Spironolactone is an anti-androgen that works by reducing levels of androgens that contribute to hair loss in women, particularly in cases of androgenetic alopecia. However, the systemic side effects of spironolactone when administered orally restrict its clinical application. When administered orally, spironolactone rapidly metabolizes in the liver into a number of metabolites including canrenone. Spironolactone and its metabolites can cause diuretic effects, menstrual irregularity, breast tenderness and enlargement, dizziness, headaches, nausea and vomiting in women. In men, oral administration of spironolactone and its metabolites can cause a decrease of libido, impotence and gynecomastia. Because of this, spironolactone is currently prescribed off-label to females for acne but is not prescribed to males. Additionally, oral administration of the recommended dosage of 50 mg-100 mg spironolactone twice a day for the treatment of acne exposes patients to systemic adverse events for an indication that is local.

In order to bypass the hepatic first pass metabolism of spironolactone and prevent the adverse effects of spironolactone and its metabolites that occur when spironolactone is administered orally, topical administration for the delivery of spironolactone has been attempted. For example, U.S. Pat. No. 4,543,351 reports a spironolactone-containing composition for combating acne in the form of a cream to be applied to human skin. U.S. Pat. No. 8,003,690 describes a topical nanoparticulate spironolactone formulation comprising nanoparticles having a mean diameter in the range of from about 300 nm to about 900 nm. A 5% spironolactone topical lotion and cream have been used to treat grade II acne (Kim, 2012). Sebum secretion was treated with a 5% spironolactone gel (Yamamoto, 1996). However, creams, gels and lotions can be difficult to apply, are inconvenient, and can involve a messy application process and are known for inadvertent transfer of spironolactone to others who may come in contact with these formulations. Additionally, spironolactone and minoxidil exert challenges when being formulated, including stability challenges, permeation challenges and intrinsic solubility of the spironolactone and minoxidil in solvents. Further, none of the previous treatment methods have been demonstrated to be effective and safe in treating acne in both men and women.

### SUMMARY OF THE INVENTION

The present disclosure is directed to a liquid composition comprising about 1% w/v to about 20% w/v spironolactone and/or canrenone, about 1% w/v to about 20% w/v minoxidil, about 10% w/v to about 60% w/v aliphatic solvents (e.g., C₄-C₁₄ aliphatic solvents), a penetration enhancer, about 1% w/v to about 30% w/v polyethylene glycol, wherein the composition does not comprise C₁-C₃ alcohols.

In one embodiment, the composition comprises spironolactone. In some embodiments, the composition comprises about 1% w/v to about 10% w/v spironolactone and 1% w/v to about 10% w/v minoxidil. In some embodiments, the composition comprises about 5% w/v spironolactone and about 5% w/v of minoxidil.

In some embodiments, the aliphatic solvent is di-isopropyl adipate, dimethyl isosorbide, dimethyl sulphoxide, ethyl acetate, dimethyl isosorbide, or combinations thereof. In some embodiments, the aliphatic solvent is about 15% w/v to about 60% w/v ethyl acetate, about 10% w/v to about 65% w/v di-isopropyl adipate, and about 5% w/v to about 60% w/v dimethyl isosorbide.

In some embodiments, the penetration enhancer is azone, isopropyl myristate, octisalate, oleic acid, Transcutol® P or combinations thereof. In some embodiments, the penetration enhancer is about 1% to about 30% w/v of the composition. In some embodiments, the penetration enhancer is about 1% to about 10% w/v of the composition. In some embodiments, the penetration enhancer is oleic acid.

In some embodiments, the polyethylene glycol is polyethylene glycol 400.

In some embodiments, the composition further comprises a buffer. In some embodiments, the buffer comprises sodium citrate, citric acid buffer, or combinations thereof.

In some embodiments, the composition further comprises an emollient, humectant and/or a conditioner. In some embodiments, the emollient is cetearyl alcohol or oleic acid. In some embodiments, the humectant is glycerol.

In some embodiments, the composition further comprises an additional active agent. In some embodiments, the additional active agent is finasteride, dutasteride, niacinamide or combinations thereof. In some embodiments, the additional active agent is about 0.1% w/w to about 1.0% w/w finasteride, about 0.1% w/w to about 1.0% w/w dutasteride, or about 2% w/v to about 10% w/v niacinamide.

In some embodiments, the pH of the composition is about 4 to about 6. In some embodiments, the pH of the composition is about 4.5. In some embodiments, the composition has a viscosity of 100-2000 cPs at room temperature.

In some embodiments, the composition comprises an antioxidant, a preservative and/or a fragrance.

In some embodiments, the composition is non-irritant when applied to the scalp.

In some embodiments, the spironolactone and minoxidil do not crystallize when the composition is applied to the skin surface.

In some embodiments, the composition is in the form of a solution, cream, gel, foam or a spray.

In some embodiments, the disclosure provides a method of treating hair loss in a subject, the method comprising topically applying the composition to a scalp surface of the subject. In some embodiments, the composition is applied only once within a 24 hour period.

In some embodiments, the composition is applied at least as a first application at a first timepoint and a second application at a second timepoint, wherein the second timepoint is at least 24 hours after the first timepoint.

In some embodiments, the composition is applied at three or more applications at three or more timepoints. In some embodiments, the time between each timepoint is about 24 hours.

In some embodiments, the first application and the second application are applied at the same site on the skin surface of the subject. In some embodiments, the first application and the second application are applied at different sites on the skin surface of the subject.

In some embodiments, the subject is human. In some embodiments, the subject is female.

In some embodiments, the composition is applied to the skin surface in a single actuation in a volume of about 1 mL or less.

In one embodiment, the present disclosure is directed to a spray container comprising the any one of the compositions disclosed herein and a metering valve. In some embodiments, the container further comprises a dose indicator. In some embodiments, the container is pressurized. In some embodiments, the container further comprises a propellent.

In embodiment, the present disclosure is a dual chamber device comprising a first chamber comprising a first composition comprising 1% w/w to 10% w/w spironolactone, about 15% w/w to 20% w/w ethyl acetate and about 10% di-isopropyl adipate and a second chamber comprising a second composition comprising 1% w/w to 10% w/w of minoxidil and comprising about 40% w/w to about 50% w/w dimethyl Isosorbide, about 4% to 7% Transcutol® P, about 5% w/v to about 7% w/v isopropyl myristate and about 5.0 w/w polyethylene glycol 400, wherein the dual chamber device is suitable for providing a spray comprising the composition.

In some embodiments, the contents of the first and second chambers are mixed during spraying of the composition onto the scalp.

### DESCRIPTION OF THE INVENTION

The present disclosure relates to a liquid composition comprising spironolactone or canrenone and minoxidil and methods suitable for the treatment of male and female pattern hair loss, wherein the composition does not comprise C₁-C₃ alcohols. In some embodiments, the disclosure provides a liquid composition comprising a combination of spironolactone and minoxidil. Spironolactone and minoxidil compositions have been used previously in the forms of topical lotions, sprays and gels. However, the present disclosure provides for a new composition comprising both spironolactone and minoxidil that do not contain ethanol or propylene glycol, which dry out the skin and cause the irritation implicated with minoxidil formulations, and that can be applied and rubbed into hair without leaving behind a residue. The present disclosure provides a liquid composition suitable for application to the skin without undue irritation, while maintaining the physical stability, chemical stability and efficacy of the spironolactone (and/or canrenone) and minoxidil. Applicant has found a liquid composition in which the spironolactone (and/or canrenone) and minoxidil remains stable over an extended period of time, while still allowing for topical penetration of spironolactone (and/or canrenone) and minoxidil within the skin. The present disclosure also provides for a liquid composition that does not contain C₁-C₃ alcohols and in which the spironolactone and minoxidil does not precipitate when stored for an extended period of time.

In some embodiments, the liquid compositions described herein can be sprayed directly on the skin surface, and do not need to be spread over the skin by touching with fingers or with use of a separate applicator. In some embodiments, drug absorption into the skin is rapid and occurs in less than four hours. In some embodiments, during solvent evaporation, the excipients included in the sprayable liquid compositions can prevent recrystallization of spironolactone, canrenone and minoxidil, a feature that can be important for its penetration of the skin or scalp. In some embodiments, the spironolactone, canrenone and minoxidil in the liquid composition described herein do not crystallize when the composition is applied to the skin. In some embodiments, direct application to the skin via a liquid composition that is sprayed provides for increased convenience, and lower transmission of the spironolactone canrenone and minoxidil to the hands/fingers or other body parts for which it is not needed. In some embodiments, the sprayable liquid compositions described herein can be applied quickly. In some embodiments, the sprayable liquid compositions described herein can be applied quickly over a large area of the skin or scalp.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. A used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.,” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

As used herein, “room temperature” is an indoor temperature suitable for long term storage of biological matter and laboratory experimentation, typically ranging between 15-28 °C. In embodiments, room temperature is from 20-25 °C.

The present disclosure is directed to a liquid composition comprising about 1% w/v to about 20% w/v spironolactone and/or canrenone, about 1% w/v to about 20% w/v minoxidil, about 10% w/v to about 60% w/v aliphatic solvents (e.g., C₄-C₁₄ aliphatic solvents), a penetration enhancer, and about 1% w/v to about 30% w/v polyethylene glycol, wherein the composition does not comprise C₁-C₃ alcohols. In some embodiments, the composition comprises spironolactone.

Various concentrations of minoxidil, spironolactone and canrenone can be used in the composition. In some embodiments, the composition comprises about 1% w/v to about 20% w/v minoxidil, spironolactone and/or canrenone. In some embodiments, the composition comprises about 1% w/v to about 19% w/v minoxidil, spironolactone and/or canrenone. In some embodiments, the composition comprises about 1% w/v to about 18% w/v minoxidil, spironolactone and/or canrenone. In some embodiments, the composition comprises about 1% w/v to about 17% w/v minoxidil, spironolactone and/or canrenone. In some embodiments, the composition comprises about 1% w/v to about 16% w/v minoxidil, spironolactone and/or canrenone. In some embodiments, the composition comprises about 1% w/v to about 15% w/v minoxidil, spironolactone and/or canrenone. In some embodiments, the composition comprises about 1% w/v to about 14% w/v minoxidil, spironolactone and/or canrenone. In some embodiments, the composition comprises about 1% w/v to about 13% w/v minoxidil, spironolactone and/or canrenone. In some embodiments, the composition comprises about 1% w/v to about 12% w/v minoxidil, spironolactone and/or canrenone. In some embodiments, the composition comprises about 1% w/v to about 11% w/v minoxidil, spironolactone and/or canrenone. In some embodiments, the composition comprises about 1% w/v to about 10% w/v minoxidil, spironolactone and/or canrenone. In some embodiments, the composition comprises about 1% w/v to about 9% w/v minoxidil, spironolactone and/or canrenone. In some embodiments, the composition comprises about 1% w/v to about 8% w/v minoxidil, spironolactone and/or canrenone. In some embodiments, the composition comprises about 1% w/v to about 7% w/v minoxidil, spironolactone and/or canrenone. In some embodiments, the composition comprises about 1% w/v to about 6% w/v minoxidil, spironolactone and/or canrenone. In some embodiments, the composition comprises about 1% w/v to about 5% w/v minoxidil, spironolactone and/or canrenone. In some embodiments, the composition comprises about 1% w/v to about 4% w/v minoxidil, spironolactone and/or canrenone. In some embodiments, the composition comprises about 1% w/v to about 3% w/v minoxidil, spironolactone and/or canrenone.

In some embodiments, the composition comprises about 2% w/v to about 20% w/v minoxidil, spironolactone and/or canrenone. In some embodiments, the composition comprises about 2% w/v to about 19% w/v minoxidil, spironolactone and/or canrenone. In some embodiments, the composition comprises about 2% w/v to about 18% w/v minoxidil, spironolactone and/or canrenone. In some embodiments, the composition comprises about 2% w/v to about 17% w/v minoxidil, spironolactone and/or canrenone. In some embodiments, the composition comprises about 2% w/v to about 16% w/v minoxidil, spironolactone and/or canrenone. In some embodiments, the composition comprises about 2% w/v to about 15% w/v minoxidil, spironolactone and/or canrenone. In some embodiments, the composition comprises about 2% w/v to about 14% w/v minoxidil, spironolactone and/or canrenone. In some embodiments, the composition comprises about 2% w/v to about 13% w/v minoxidil, spironolactone and/or canrenone. In some embodiments, the composition comprises about 2% w/v to about 12% w/v minoxidil, spironolactone and/or canrenone. In some embodiments, the composition comprises about 2% w/v to about 11% w/v minoxidil, spironolactone and/or canrenone. In some embodiments, the composition comprises about 2% w/v to about 10% w/v minoxidil, spironolactone and/or canrenone. In some embodiments, the composition comprises about 2% w/v to about 9% w/v minoxidil, spironolactone and/or canrenone. In some embodiments, the composition comprises about 2% w/v to about 8% w/v minoxidil, spironolactone and/or canrenone. In some embodiments, the composition comprises about 2% w/v to about 7% w/v minoxidil, spironolactone and/or canrenone. In some embodiments, the composition comprises about 2% w/v to about 6% w/v minoxidil, spironolactone and/or canrenone. In some embodiments, the composition comprises about 2% w/v to about 5% w/v minoxidil and spironolactone. In some embodiments, the composition comprises about 2% w/v to about 4% w/v minoxidil, spironolactone and/or canrenone. In some embodiments, the composition comprises about 2% w/v to about 3% w/v minoxidil, spironolactone and/or canrenone.

In some embodiments, the composition comprises about 3% w/v to about 20% w/v minoxidil, spironolactone and/or canrenone. In some embodiments, the composition comprises about 3% w/v to about 19% w/minoxidil, spironolactone and/or canrenone. In some embodiments, the composition comprises about 3% w/v to about 18% w/v minoxidil, spironolactone and/or canrenone. In some embodiments, the composition comprises about 3% w/v to about 17% w/v minoxidil, spironolactone and/or canrenone In some embodiments, the composition comprises about 3% w/v to about 16% w/v minoxidil, spironolactone and/or canrenone. In some embodiments, the composition comprises about 3% w/v to about 15% w/v minoxidil, spironolactone and/or canrenone. In some embodiments, the composition comprises about 3% w/v to about 14% w/v minoxidil, spironolactone and/or canrenone. In some embodiments, the composition comprises about 3% w/v to about 13% w/v minoxidil, spironolactone and/or canrenone. In some embodiments, the composition comprises about 3% w/v to about 12% w/v minoxidil, spironolactone and/or canrenone. In some embodiments, the composition comprises about 3% w/v to about 11% w/v minoxidil, spironolactone and/or canrenone. In some embodiments, the composition comprises about 3% w/v to about 10% w/v minoxidil, spironolactone and/or canrenone. In some embodiments, the composition comprises about 3% w/v to about 9% w/v minoxidil, spironolactone and/or canrenone. In some embodiments, the composition comprises about 3% w/v to about 8% w/v minoxidil, spironolactone and/or canrenone. In some embodiments, the composition comprises about 3% w/v to about 7% w/v minoxidil, spironolactone and/or canrenone. In some embodiments, the composition comprises about 3% w/v to about 6% w/v minoxidil, spironolactone and/or canrenone. In some embodiments, the composition comprises about 3% w/v to about 5% w/v minoxidil, spironolactone and/or canrenone. In some embodiments, the composition comprises about 3% w/v to about 4% w/v minoxidil, spironolactone and/or canrenone.

In some embodiments, the composition comprises about 4% w/v to about 20% w/v minoxidil, spironolactone and/or canrenone. In some embodiments, the composition comprises about 4% w/v to about 19% w/v minoxidil, spironolactone and/or canrenone. In some embodiments, the composition comprises about 4% w/v to about 18% w/v minoxidil, spironolactone and/or canrenone. In some embodiments, the composition comprises about 4% w/v to about 17% w/v minoxidil, spironolactone and/or canrenone. In some embodiments, the composition comprises about 4% w/v to about 16% w/v minoxidil, spironolactone and/or canrenone. In some embodiments, the composition comprises about 4% w/v to about 15% w/v minoxidil, spironolactone and/or canrenone. In some embodiments, the composition comprises about 4% w/v to about 14% w/v minoxidil, spironolactone and/or canrenone. In some embodiments, the composition comprises about 4% w/v to about 13% w/v minoxidil, spironolactone and/or canrenone. In some embodiments, the composition comprises about 4% w/v to about 12% w/v minoxidil, spironolactone and/or canrenone. In some embodiments, the composition comprises about 4% w/v to about 11% w/v minoxidil, spironolactone and/or canrenone. In some embodiments, the composition comprises about 4% w/v to about 10% w/v minoxidil, spironolactone and/or canrenone. In some embodiments, the composition comprises about 4% w/v to about 9% w/v minoxidil, spironolactone and/or canrenone. In some embodiments, the composition comprises about 4% w/v to about 8% w/v minoxidil, spironolactone and/or canrenone. In some embodiments, the composition comprises about 4% w/v to about 7% w/v minoxidil, spironolactone and/or canrenone. In some embodiments, the composition comprises about 4% w/v to about 6% w/v minoxidil, spironolactone and/or canrenone. In some embodiments, the composition comprises about 4% w/v to about 5% w/v minoxidil, spironolactone and/or canrenone.

In some embodiments, the composition comprises about 5% w/v to about 20% w/v minoxidil, spironolactone and/or canrenone. In some embodiments, the composition comprises about 5% w/v to about 19% w/v minoxidil, spironolactone and/or canrenone. In some embodiments, the composition comprises about 5% w/v to about 18% w/v minoxidil, spironolactone and/or canrenone. In some embodiments, the composition comprises about 5% w/v to about 17% w/v minoxidil, spironolactone and/or canrenone. In some embodiments, the composition comprises about 5% w/v to about 16% w/v minoxidil, spironolactone and/or canrenone. In some embodiments, the composition comprises about 5% w/v to about 15% w/v minoxidil, spironolactone and/or canrenone. In some embodiments, the composition comprises about 5% w/v to about 14% w/minoxidil, spironolactone and/or canrenone. In some embodiments, the composition comprises about 5% w/v to about 13% w/v minoxidil, spironolactone and/or canrenone. In some embodiments, the composition comprises about 5% w/v to about 12% w/v minoxidil, spironolactone and/or canrenone. In some embodiments, the composition comprises about 5% w/v to about 11% w/v minoxidil, spironolactone and/or canrenone. In some embodiments, the composition comprises about 5% w/v to about 10% w/v minoxidil, spironolactone and/or canrenone. In some embodiments, the composition comprises about 5% w/v to about 9% w/v minoxidil, spironolactone and/or canrenone. In some embodiments, the composition comprises about 5% w/v to about 8% w/v minoxidil, spironolactone and/or canrenone. In some embodiments, the composition comprises about 5% w/v to about 7% w/v minoxidil, spironolactone and/or canrenone. In some embodiments, the composition comprises about 5% w/v to about 6% w/v minoxidil, spironolactone and/or canrenone.

In some embodiments, the composition comprises about 6% w/v to about 20% w/v minoxidil, spironolactone and/or canrenone. In some embodiments, the composition comprises about 6% w/v to about 19% w/v minoxidil, spironolactone and/or canrenone. In some embodiments, the composition comprises about 6% w/v to about 18% w/v minoxidil, spironolactone and/or canrenone. In some embodiments, the composition comprises about 6% w/v to about 17% w/v minoxidil, spironolactone and/or canrenone. In some embodiments, the composition comprises about 6% w/v to about 16% w/v minoxidil, spironolactone and/or canrenone. In some embodiments, the composition comprises about 6% w/v to about 15% w/v minoxidil, spironolactone and/or canrenone. In some embodiments, the composition comprises about 6% w/v to about 14% w/v minoxidil, spironolactone and/or canrenone. In some embodiments, the composition comprises about 6% w/v to about 13% w/v minoxidil, spironolactone and/or canrenone. In some embodiments, the composition comprises about 6% w/v to about 12% w/v minoxidil, spironolactone and/or canrenone. In some embodiments, the composition comprises about 6% w/v to about 11% w/v minoxidil, spironolactone and/or canrenone. In some embodiments, the composition comprises about 6% w/v to about 10% w/v minoxidil, spironolactone and/or canrenone. In some embodiments, the composition comprises about 6% w/v to about 9% w/v minoxidil, spironolactone and/or canrenone. In some embodiments, the composition comprises about 6% w/v to about 8% w/v minoxidil, spironolactone and/or canrenone. In some embodiments, the composition comprises about 6% w/v to about 7% w/v minoxidil, spironolactone and/or canrenone.

In some embodiments, the composition comprises about 7% w/v to about 20% w/v minoxidil, spironolactone and/or canrenone. In some embodiments, the composition comprises about 7% w/v to about 19% w/v minoxidil, spironolactone and/or canrenone. In some embodiments, the composition comprises about 7% w/v to about 18% w/v minoxidil, spironolactone and/or canrenone. In some embodiments, the composition comprises about 7% w/v to about 17% w/v minoxidil, spironolactone and/or canrenone. In some embodiments, the composition comprises about 7% w/v to about 16% w/v minoxidil, spironolactone and/or canrenone. In some embodiments, the composition comprises about 7% w/v to about 15% w/v minoxidil, spironolactone and/or canrenone. In some embodiments, the composition comprises about 7% w/v to about 14% w/v minoxidil, spironolactone and/or canrenone. In some embodiments, the composition comprises about 7% w/v to about 13% w/v minoxidil, spironolactone and/or canrenone. In some embodiments, the composition comprises about 7% w/v to about 12% w/v minoxidil, spironolactone and/or canrenone. In some embodiments, the composition comprises about 7% w/v to about 11% w/v minoxidil, spironolactone and/or canrenone. In some embodiments, the composition comprises about 7% w/v to about 10% w/v minoxidil, spironolactone and/or canrenone. In some embodiments, the composition comprises about 7% w/v to about 9% w/v minoxidil, spironolactone and/or canrenone. In some embodiments, the composition comprises about 7% w/v to about 8% w/v minoxidil, spironolactone and/or canrenone.

In some embodiments, the composition comprises about 8% w/v to about 20% w/v minoxidil, spironolactone and/or canrenone. In some embodiments, the composition comprises about 8% w/v to about 19% w/v minoxidil, spironolactone and/or canrenone. In some embodiments, the composition comprises about 8% w/v to about 18% w/v minoxidil, spironolactone and/or canrenone. In some embodiments, the composition comprises about 8% w/v to about 17% w/v minoxidil, spironolactone and/or canrenone. In some embodiments, the composition comprises about 8% w/v to about 16% w/v minoxidil, spironolactone and/or canrenone. In some embodiments, the composition comprises about 8% w/v to about 15% w/v minoxidil, spironolactone and/or canrenone. In some embodiments, the composition comprises about 8% w/v to about 14% w/v minoxidil, spironolactone and/or canrenone. In some embodiments, the composition comprises about 8% w/v to about 13% w/v minoxidil, spironolactone and/or canrenone. In some embodiments, the composition comprises about 8% w/v to about 12% w/v minoxidil, spironolactone and/or canrenone. In some embodiments, the composition comprises about 8% w/v to about 11% w/v minoxidil, spironolactone and/or canrenone. In some embodiments, the composition comprises about 8% w/v to about 10% w/v minoxidil, spironolactone and/or canrenone. In some embodiments, the composition comprises about 8% w/v to about 9% w/v minoxidil, spironolactone and/or canrenone.

In some embodiments, the composition comprises about 9% w/v to about 20% w/v minoxidil, spironolactone and/or canrenone. In some embodiments, the composition comprises about 9% w/v to about 19% w/v minoxidil, spironolactone and/or canrenone. In some embodiments, the composition comprises about 9% w/v to about 18% w/v minoxidil, spironolactone and/or canrenone. In some embodiments, the composition comprises about 9% w/v to about 17% w/v minoxidil, spironolactone and/or canrenone. In some embodiments, the composition comprises about 9% w/v to about 16% w/v minoxidil, spironolactone and/or canrenone. In some embodiments, the composition comprises about 9% w/v to about 15% w/v minoxidil, spironolactone and/or canrenone. In some embodiments, the composition comprises about 9% w/v to about 14% w/v minoxidil, spironolactone and/or canrenone. In some embodiments, the composition comprises about 9% w/v to about 13% w/v minoxidil, spironolactone and/or canrenone. In some embodiments, the composition comprises about 9% w/v to about 12% w/v minoxidil, spironolactone and/or canrenone. In some embodiments, the composition comprises about 9% w/v to about 11% w/v minoxidil, spironolactone and/or canrenone. In some embodiments, the composition comprises about 9% w/v to about 10% w/v minoxidil, spironolactone and/or canrenone.

In some embodiments, the composition comprises about 10% w/v to about 20% w/v minoxidil, spironolactone and/or canrenone. In some embodiments, the composition comprises about 10% w/v to about 19% w/v minoxidil, spironolactone and/or canrenone. In some embodiments, the composition comprises about 10% w/v to about 18% w/v minoxidil, spironolactone and/or canrenone. In some embodiments, the composition comprises about 10% w/v to about 17% w/v minoxidil, spironolactone and/or canrenone. In some embodiments, the composition comprises about 10% w/v to about 16% w/v minoxidil, spironolactone and/or canrenone. In some embodiments, the composition comprises about 10% w/v to about 15% w/v minoxidil, spironolactone and/or canrenone. In some embodiments, the composition comprises about 10% w/v to about 14% w/v minoxidil, spironolactone and/or canrenone. In some embodiments, the composition comprises about 10% w/v to about 13% w/v minoxidil, spironolactone and/or canrenone. In some embodiments, the composition comprises about 10% w/v to about 12% w/v minoxidil, spironolactone and/or canrenone. In some embodiments, the composition comprises about 10% w/v to about 11% w/v minoxidil, spironolactone and/or canrenone.

In some embodiments, the composition comprises about 1% minoxidil, spironolactone and/or canrenone. In some embodiments, the composition comprises about 2% minoxidil, spironolactone and/or canrenone. In some embodiments, the composition comprises about 3% minoxidil, spironolactone and/or canrenone. In some embodiments, the composition comprises about 4% minoxidil, spironolactone and/or canrenone. In some embodiments, the composition comprises about 5% minoxidil, spironolactone and/or canrenone. In some embodiments, the composition comprises about 6% minoxidil, spironolactone and/or canrenone. In some embodiments, the composition comprises about 7% minoxidil, spironolactone and/or canrenone. In some embodiments, the composition comprises about 8% minoxidil, and spironolactone and/or canrenone. In some embodiments, the composition comprises about 9% minoxidil, and spironolactone and/or canrenone. In some embodiments, the composition comprises about 10% minoxidil, and spironolactone and/or canrenone. In some embodiments, the composition comprises about 11% minoxidil, and spironolactone and/or canrenone. In some embodiments, the composition comprises about 12% minoxidil, and spironolactone and/or canrenone. In some embodiments, the composition comprises about 13% minoxidil, and spironolactone and/or canrenone. In some embodiments, the composition comprises about 14% minoxidil, and spironolactone and/or canrenone. In some embodiments, the composition comprises about 15% minoxidil, and spironolactone and/or canrenone. In some embodiments, the composition comprises about 16% minoxidil, and spironolactone and/or canrenone. In some embodiments, the composition comprises about 17% minoxidil, and spironolactone and/or canrenone. In some embodiments, the composition comprises about 18% minoxidil, and spironolactone and/or canrenone. In some embodiments, the composition comprises about 19% minoxidil, and spironolactone and/or canrenone. In some embodiments, the composition comprises about 20% minoxidil, and spironolactone and/or canrenone.

Various aliphatic solvents can be used in the liquid compositions of the present disclosure. The term “aliphatic solvent” enhancer refers to any solvent comprising compounds that are without a ring structure, e.g., without an aromatic ring structure. In some embodiments, the term “aliphatic solvent” refers to mixtures of saturated, long straight-chain, branched-chain, or cyclic paraffins that have low viscosity, e.g., less than 500 cP, less than 100 cP, less than 50 cP, less than 30 cP, less than 10 cP, or less than 5 cP at 20 °C, which are suitable for being applied by spraying. In some embodiments, the aliphatic solvent is suitable for solubilizing minoxidil and, spironolactone and/or canrenone and forming a stable composition, e.g., a non-aromatic solvent, which is pharmaceutically acceptable. In some embodiments, the solvent is an alcoholic aliphatic solvent. In some embodiments, the aliphatic solvent is, isooctane, propane, squalene, di-isopropyl adipate, di-methyl sulphoxide, ethyl acetate, and polyethylene. In some embodiments, aliphatic solvents can include more than one solvent, e.g., a mixture of aliphatic solvents. In some embodiments, the solvent is ethyl acetate or a mixture of ethyl acetate with other aliphatic solvents. In some embodiments, the aliphatic solvent has a viscosity suitable for administering via an aerosol spray or a mist. In some embodiments, the aliphatic solvent can be used to solubilize the liquid compositions described herein, i.e., the liquid compositions are a solution.

In some embodiments, the compositions comprise less than 1% ethanol, propanol, isopropanol, propylene glycol, or combinations thereof. In some embodiments, the compositions does not comprise ethanol, propanol, isopropanol, or propylene glycol.

In some embodiments, the aliphatic solvent comprises two solvents at a ratio of about 1:100 to about 100:1. In some embodiments, the aliphatic solvent comprises two solvents at a ratio of about 5:95 to about 95:5. In some embodiments, the aliphatic solvent comprises two solvents at a ratio of about 20:80 to about 80:20. In some embodiments, the aliphatic solvent comprises two solvents at ratio of about 30:70 to about 70:30. In some embodiments, the aliphatic solvent comprises two solvents at a ratio of about 40:60 to about 60:40. In some embodiments, the aliphatic solvent comprises two solvents at a ratio of about 50:50.

In some embodiments, the aliphatic solvent comprises three solvents at a ratio of about 1:1:1 to about 1:1:10. In some embodiments, the aliphatic solvent comprises three solvents at a ratio of about 1:2:2 to about 1:2:4. In some embodiments, the aliphatic solvent comprises three solvents at a ratio of about 1:3:1 to about 1:3:6. In some embodiments, the aliphatic solvent comprises three solvents at a ratio of about 1:4:1 to about 1:4:12.

The total aliphatic solvent concentration can be around 30% to about 97% weight of the composition, but in some embodiments the aliphatic solvent is in a sufficient quantity to dissolve the other excipients and spironolactone and/or minoxidil. In some embodiments, the composition comprises about 65% weight aliphatic solvent. In some embodiments, the composition comprises about 50% to about 90% by weight aliphatic solvent. In some embodiments, the composition comprises about 30% to about 90%, about 40% to about 90%, about 60% to about 90% or about 70% to about 90% by weight aliphatic solvent. In some embodiments, the composition comprises about 52% to about 68%, about 54% to about 66%, about 56% to about 64% or about 58% to about 62% by weight aliphatic solvent. In some embodiments, the composition comprises about 35% to about 45% by weight aliphatic solvent. In some embodiments, the composition comprises about 70% by weight aliphatic solvent. In some embodiments, the composition comprises less than 80%, less than 70%, less than 65%, less than 62% or less than 61% by weight of aliphatic solvent. In some embodiments, the composition comprises about 70% to about 85% aliphatic solvent, about 72% to about 82% aliphatic solvent, or about 74% to about 80% aliphatic solvent by weight. In some embodiments, the composition comprises about 75%, about 76%, about 77%, about 78%, about 79%, about 80%, about 81%, about 82% or about 83% aliphatic solvent by weight. In some embodiments, the reduced aliphatic solvent concentration can result in reduced adverse effect, e.g., inflammation or irritation.

In some embodiments, the aliphatic solvent comprises ethyl acetate, di-isopropyl adipate, and dimethyl isosorbide. In some embodiments, the ethyl acetate, di-isopropyl adipate, and dimethyl isosorbide are in a ratio of about 1:1:1 to about 1:1:10. In some embodiments, the ethyl acetate, di-isopropyl adipate, and dimethyl isosorbide are in a ratio of about 1:2:2 to about 1:2:4. In some embodiments, the ethyl acetate, di-isopropyl adipate, and dimethyl Isosorbide are in a ratio of about 1:3:1 to about 1:3:6. In some embodiments, the ethyl acetate, di-isopropyl adipate, and dimethyl isosorbide are in a ratio of about 1:4:1 to about 1:4:12.

In some embodiments, the liquid composition is about 15% w/v to about 60% w/v ethyl acetate, about 10% w/v to about 65% w/v di-isopropyl adipate, and about 5% w/v to about 60% w/v dimethyl isosorbide. In some embodiments, the sprayable liquid composition is about 25% w/v to about 50% w/v ethyl acetate, about 20% w/v to about 55% w/v di-isopropyl adipate, and about 10% w/v to about 50% w/v dimethyl isosorbide. In some embodiments, the sprayable liquid composition is about 30% w/v to about 40% w/v ethyl acetate, about 30% w/v to about 40% w/v di-isopropyl adipate, and about 30% w/v to about 50% w/v dimethyl isosorbide. In some embodiments, when the liquid sprayable composition is applied to the skin, the solvent is volatile and evaporates (i.e., dries), leaving a trace amount (or less) of the solvent on the skin. In some embodiments, the aliphatic solvent is a volatile solvent that evaporates (i.e., dries) within one minute or in less than 30 seconds after the composition is sprayed onto a surface at room temperature and 1 atmospheric pressure.

Applicant has found that the liquid composition described in the present disclosure provides for a chemically stable formulation, wherein the liquid composition has reduced impurities over an extended period of time. The term “chemically stable” indicates the spironolactone and minoxidil does not appreciably degrade to form impurities in the liquid composition. Various degradation impurities of spironolactone and minoxidil are known in the art. Degradation impurities of spironolactone include (a) a Spironolactone EP Impurity E (7β-Spironolactone) (b) Spironolactone EP Impurity D (Disulfanyl-spironolactone), (c) a Spironolactone impurity H impurity chemical formula (C₂₀H2602) and (d) a canrenone impurity. Degradation impurities of minoxidil include (a) a Minoxidil-Impurity A: 6-Chloro-pyrimidine-2,4-diamine 3-Oxide, (b) Minoxidil-Impurity B: 6-Chloro-pyrimidine-2,4-diamine, (c) Minoxidil-Impurity C: 3-(cyanoimino)-3-(piperidin-1-yl) propenamide, (d) Minoxidil-Impurity D: 6-[[(4-methylphenyl) sulphonyl]-oxy]pyrimidine-2,4-diamine 3-oxide, (e) Minoxidil E (Freebase): Desoxyminoxidil, and (f) a 2,6-diaminopyrimidine-4-yl acetate impurity.

The term “physically stable” can refer to particles comprising minoxidil, spironolactone and/or canrenone that remain homogenously dispersed in the liquid composition for an extended period of time, i.e., the particles do not precipitate. Likewise, the term “physically stable” can refer to particles that remain homogenously dispersed in the liquid composition for an extended period of time, i.e., the particles do not form aggregates in the composition. The term “physically stable” can also refer to a liquid composition in which no phase separation is identified for an extended period of time. Additionally, the term “physically stable” can refer to particles that remain the same size distribution in the liquid composition for an extended period of time, i.e., the particles do not further crystalize, agglomerate, dissolve, or otherwise break apart in the composition.

In some embodiments, the particles comprising minoxidil, spironolactone and/or canrenone are physically stable an “extended period of time.” One skilled in the art can appreciate the benefits of a liquid composition that is stable during manufacturing, transport and storage of the liquid composition before use. In some embodiments, extended period of time can include for at least one week, at least two weeks, at least three weeks, at least one month, at least two months, three months, at least four months, at least six months, at least one year or at least two years.

The skilled artisan can appreciate that physical stability can be affected by several factors, e.g., temperature, humidity, oxidation, sunlight, etc. As used herein, physical stability is determined using standard conditions or accelerated conditions in a sealed container not subject to light degradation or oxidation.

In some embodiments, the particle size of particles in the composition comprising spironolactone and minoxidil in the composition does not change greater than 20% over a period of three months at 25 °C, six months at 25 °C, nine months at 25 °C, or one year at 25 °C. In some embodiments, the particle size in the composition does not change greater than 10% over a period of three months at 25 °C, six months at 25 °C, nine months at 25 °C, or one year at 25 °C. In some embodiments, the particle size in the composition does not change greater than 5% over a period of three months at 25 °C, six months at 25 °C, nine months at 25 °C, or one year at 25 °C. In some embodiments, the particle size in the composition does not change greater than 1% over a period of three months at 25 °C, six months at 25 °C, nine months at 25 °C, or one year at 25 °C.

In some embodiments, the particle size of particles comprising spironolactone and minoxidil in the composition does not change greater than 20% over a period of three months at 37 °C, six months at 37 °C, nine months at 37 °C, or one year at 37 °C. In some embodiments, the particle size in the composition does not change greater than 10% over a period of three months at 37 °C, six months at 37 °C, nine months at 37 °C, or one year at 37 °C. In some embodiments, the particle size in the composition does not change greater than 5% over a period of three months at 37 °C, six months at 37 °C, nine months at 37 °C, or one year at 37 °C. In some embodiments, the particle size in the composition does not change greater than 1% over a period of three months at 37 °C, six months at 37 °C, nine months at 37 °C, or one year at 37 °C.

Oxygen sensitivity in drugs and their formulations is a large concern in pharmaceutical development. Often, oxygen sensitive drug or formulation requires additional excipients, packaging and/or manufacturing steps for stability enhancement and degradation prevention. In some embodiments, the liquid compositions comprising spironolactone and minoxidil of the present disclosure have enhanced physical stability, and wherein deterioration from the presence of oxygen and/or water is minimized and/or controlled, to yield a stable formulation. In some embodiments, the oxygen content in the composition is less than 40%, less than 30%, less than 20%, less than 10%, less than 5% or less than 2%.

In some embodiments, the liquid compositions of the present disclosure comprises water. In some embodiments, the composition comprises about 1% to about 10%, about 1% to about 9%, about 1% to about 8%, about 1% to about 7%, about 1% to about 6%, or about 1% to about 5% v/v water. In some embodiments, the composition comprises about 2% to about 10%, about 2% to about 9%, about 2% to about 8%, about 2% to about 7%, about 2% to about 6%, or about 2% to about 5% v/v water. In some embodiments, the composition comprises about 3% to about 10%, about 3% to about 9%, about 3% to about 8%, about 3% to about 7%, about 3% to about 6%, or about 3% to about 5% v/v water. In some embodiments, the composition comprises about 4% to about 10%, about 4% to about 9%, about 4% to about 8%, about 4% to about 7%, about 4% to about 6%, or about 4% to about 5% v/v water. In some embodiments, the reduced water concentration decreases the drying time of the sprayable liquid compositions described herein.

In some embodiments, the liquid composition can comprise a penetration enhancer. The term “penetration enhancer” refers to any compound or composition that penetrate into the skin and interact with skin constituents to promote drug flux or reversibly decrease the barrier resistance. Penetration enhancers can include substances which promote the dermal absorption of spironolactone and/or minoxidil through the skin temporarily by transiently disturbing the lipid bilayer structure in the stratum corneum barrier or enhancing the solubility of the drug in the skin to facilitate drug delivery. In some embodiments, the penetration enhancer can include any penetration enhancer known to be pharmaceutically acceptable. In some embodiments, the penetration enhancer can include any skin enhancer as found in the U.S. Pharmacopeia, or otherwise known in the art, e.g., M. E. Lane, Skin Permeation Enhancers, Int. J. Pharma 447(1-2): 12-21 (2013). In some embodiments, the penetration enhancer can include 1-dodecylazacycloheptan-2-one, isopropyl myristate, octisalate, oleic acid, diethylene glycol monoethyl ether (Transcutol®), or combination thereof. In some embodiments, one, two, three or greater than three penetration enhancers are on the sprayable liquid composition. In some embodiments, two penetration enhancers are in the composition. In some embodiments, the penetration enhancer is azone, isopropyl myristate, octisalate, oleic acid, Transcutol® P or combinations thereof.

Various concentrations of penetration enhancers can be present in the sprayable liquid composition. The permeation enhancer can be adjusted to facilitate the penetration of spironolactone and/or minoxidil. In some embodiments, the composition comprises about 0.1% w/v to about 20% w/v, from about 0.1% w/v to about 15% w/v, from about 0.1% w/v to about 10% w/v, about 0.1% w/v to about 5% w/v, or about 0.5% w/v to about 8% w/v penetration enhancer. In some embodiments, the composition comprises about 4% to about 25% by weight penetration enhancer. In some embodiments, the composition comprises about 4% to about 20% by weight penetration enhancer. In some embodiments, the composition comprises about 4% to about 15% by weight penetration enhancer. In some embodiments, the composition comprises about 4% to about 10% by weight penetration enhancer. In some embodiments, the composition comprises about 10% to about 15% by weight penetration enhancer. In some embodiments, the composition comprises about 10% to about 20% by weight penetration enhancer. In some embodiments, the composition comprises about 15% to about 20% by weight penetration enhancer. In some embodiments, the composition comprises about 15% to about 25% by weight penetration enhancer.

In some embodiments, the liquid composition comprises about 1% to about 10% of the penetration enhancer. In some embodiments, the composition comprises about 2% to about 10% of the penetration enhancer. In some embodiments, the composition comprises about 3% to about 10% of the penetration enhancer. In some embodiments, the composition comprises about 4% to about 10% of the penetration enhancer. In some embodiments, the composition comprises about 5% to about 10% of the penetration enhancer. In some embodiments, the composition comprises about 6% to about 10% of the penetration enhancer. In some embodiments, the composition comprises about 7% to about 10% of the penetration enhancer. In some embodiments, the composition comprises about 8% to about 10% of the penetration enhancer. In some embodiments, the composition comprises about 9% to about 10% of the penetration enhancer.

In some embodiments, the liquid compositions can be washed off via convenient methods, i.e., showering, when absorption of the drug into the scalp is complete. A washability enhancer is included in the composition to make the composition more washable, i.e., easier to remove from skin with water or soap and water. In some cases, the washability enhancer also acts as an emulsifier, emollient, conditioner and plasticizer agent to prevent hair from drying out. In some embodiments, the washability enhancer is a low molecular weight polyethylene glycol (PEG) containing ethylene glycol polymer of various molecular weights, such as polyethylene glycol 300, 400 or 600.

With regard to the total weight of the composition, in some embodiments, the concentration of washability enhancer in the composition can be about 0.1% w/v to about 50% w/v, about 0.1% w/v to about 30% w/v, about 0.1% w/v to about 15% w/v, about 0.1% w/v to about 5% w/v, about 0.5% w/v to about 5% w/v, or about 1% w/v to about 5% w/v of the composition.

The liquid composition can include one or more plasticizers such as dibutyl sebacate, triethyl citrate, triacetin, glycerol, a low molecular weight polyethylene glycol, e.g., polyethylene glycol 300, 400 or 600, etc. The concentration of plasticizer in the composition can be about 0.1% w/v to about 20% w/v, about 0.1% w/v to about 15% w/v, about 0.1% w/v to about 10% w/v, about 0.1% w/v to about 5% w/v, about 0.5% w/v to about 5% w/v, or about 1% w/v to about 5% w/v of the composition. In some embodiments it is about 0.5% w/v, about 1% w/v, about 1.5% w/v, about 2% w/v, about 2.5% w/v, about 3% w/v, about 3.5% w/v, or about 4% w/v, of the composition. In some embodiments, the plasticizer is also a washability enhancer.

The liquid composition can also include one or more viscosity increasing agents, e.g., povidone, glycerin, hydroxypropyl cellulose, methylcellulose, and/or carboxymethylcellulose. The concentration of the viscosity increasing agent, if included, can be from about 0.1% w/v to about 10% w/v, about 0.1% w/v to about 8% w/v, about 0.1% w/v to about 5% w/v of the composition, e.g., about 0.5% w/v, about 1% w/v, about 1.5% w/v, about 2% w/v, or about 2.5% w/v of the composition. In some embodiments, the viscosity increasing agent is in an amount that allows the composition to still be a sprayable liquid solution. For example, in some embodiments, the viscosity of the composition is less than 40 mPas, less than 30 mPas, less than 20 mPas, less than 10 mPas or less than 5 mPas at room temperature and 1 atmospheric pressure.

In some embodiments, the liquid compositions can comprise viscosity increasing agents. In some embodiments, the viscosity increasing agent is a bio adhesive. Such agents include Carbopol®. Carbopol® polymers are high molecular weight, crosslinked polyacrylic acid polymers. The polymers differ by crosslink density and can be grouped into the following categories: (1) Carbopol® homopolymers, such as acrylic acid crosslinked with allyl sucrose or allyl pentaerythritol, e.g., Carbopol® 71G NF (viscosity 4,000-11,000), 971P NF (viscosity 4,000-11,000), 974P NF, (viscosity 29,400-39,400), 980 NF (viscosity 40,000-60,000), 981 NF (viscosity 4000-10,000), 5984 EP (viscosity 30,500-39,400), 934 NF (viscosity 30,500-39,400), 934P NF (viscosity 29,400-39,400), 940 NF (viscosity 40,000-60,000), 941 NF (viscosity 4,000-10,000); and (2) Carbopol® copolymers: acrylic acid and C₁₀-C₃₀ alkyl acrylate crosslinked with allyl pentaerythritol, e.g., Carbopol® 1342 NF, viscosity 9,500-26,500.

The compositions of the present disclosure can also include other excipients, added, e.g., to achieve a desired consistency or appearance, or to protect the composition components from degradation and oxidation. Such excipients include, for example, cosolvents, stabilizing agents, antioxidants, humectants, preservatives, pH modifiers, colorants, dyes, and fragrances known in the art of formulation.

Another optional excipient of the compositions is a pH modifier, such as a buffer. In some embodiments, the buffer comprises sodium citrate, citric acid, fumaric acid, monopotassium phosphate, boric acid, acetic acid, adipic acid, calcium hydroxide, glycine, hydrochloric acid, lactic acid, magnesium aluminometasilicates, phosphoric acid, sodium carbonate, sodium hydroxide, sorbic acid, succinic acid, tartaric acid, and derivatives, salts, diethyl barbituric acid or combinations thereof. In some embodiments, the buffer is present in the composition at a concentration of from 1 mM to 100 mM, about 1 mM to 50 mM, about 5 mM to 25 mM or about 10 mM. In some embodiments, the buffer is present in the composition at a concentration of 100 mM. In some embodiments, the pH of the sprayable liquid composition is about 3 to about 6, about 3.5 to about 5.5, or about 4 to about 5. In some embodiments, the pH of the sprayable liquid composition is about 4.5.

In some embodiments, the addition of emollients, emulsion stabilizers, moisturizers, excipients, and other compounds may be modified to enhance the sensory properties of the sprayable topical compositions, including but not limited to the skin feel (silkiness, lightness, creaminess, etc.), soften, soothe and moisturize the skin, support renewal of post-procedure skin, help restore the skin's moisture balance, hydrate dry and compromised skin, and is suitable for extremely dry skin. In some embodiments, the composition comprises a moisturizer. In some embodiments, the moisturizer is petrolatum, microcrystalline wax, physalis angulata extract, caprylic/capric triglyceride, butyrospermum parki (shea butter) extract, bisabolol, and tocopherol.

In some embodiments, the composition comprises an additional active agent. In some embodiments, the additional active agent is a vitamin, such as biotin for hair growth. In some embodiments, particularly for application to the scalp and hair, the composition can comprise a fragrance or perfume to impart a desired aroma, or to mask odors that can be associated with other components of the composition. In some embodiments, the concentration of the fragrance is about 0.01% w/v to about 5% w/v of the composition, or about 0.1% w/v to about 1% w/v of the composition.

Any fragrance suitable for application to the hair can be used herein including a wide variety of fragrances and perfumes that are known to those skilled in the art. In some embodiments, the amount of fragrance can be effective for providing a noticeable aroma to the composition, or for masking undesired aroma of the composition. In some embodiments, the fragrance does not impart excessive stinging to the skin, especially to broken or irritated skin.

Typical fragrances are described in Arctander, Perfume and Flavour Chemicals (Aroma Chemicals), Vol. I and II (1969, Allured Publishing Corporation, 1969 (ISBN 0931710375, 9780931710377), and Arctander, Perfume and Flavor Materials of Natural Origin (1994, by Allured Pub Corp) (ISBN 0931710367; ISBN13:9780931710360). Fragrance used in the present composition can also contain solubilizers, diluents, or solvents which are well known in the art.

High viscosity topical minoxidil, spironolactone and/or canrenone formulations, e.g., creams, lotions and gels, require application to the scalp with the palm or fingers of the hand of the user, and thus these types of formulations are associated with a significant risk for secondary minoxidil and, spironolactone and/or canrenone exposure. The present claims feature a liquid composition, i.e., low viscosity compositions, which can be applied directly to the skin in an amount effective via a cream, gel foam or spray. Such sprayable liquid composition are useful and convenient in delivering the required dosage or minoxidil and, spironolactone and/or canrenone needed for the treatment of male and female pattern hair loss. In some embodiments, the “sprayable liquid composition” has low viscosity (about 15 cPs to about 45 cPs) and can be administered in smaller volumes, i.e., in a volume less than 500 μL per actuation. In some embodiments, the sprayable liquid composition has a viscosity suitable for administering via an aerosol spray and/or a mist. For example, in some embodiments, the sprayable liquid composition has a viscosity of less than 200 cPs, less than 100 cPs, less than 50 cPs or less than 30 cPs at room temperature. In some embodiments, the sprayable liquid formation has a viscosity of about 1 cp to about 10 cPs, about 5 cPS to about 20 cPs or about 20 cPs to about 30 cPs at room temperature. In some embodiments, the sprayable liquid composition has a viscosity of about 5 cPs to about 15 cPs. As used throughout the present disclosure, viscosity is measured at standard conditions, e.g., 1 atm pressure and 25 °C temperature.

The liquid composition of the present disclosure can be administered by means to known in the art. In some embodiments, the disclosure provides a spray container for administering the composition. In some embodiments, the disclosure provides a spray container comprising the sprayable liquid compositions as described herein and a metering valve. In some embodiments, the spray container is a sealed and pressurized device, e.g., an aerosol container. In some embodiments, the spray container is a sealed and pressurized device, an aerosol container, with a means for providing an aerosol spray of the sprayable liquid composition, e.g., an aerosol nozzle. In some embodiments, the spray container further comprises a dose indicator. In some embodiments, the spray container comprises a propellant. In some embodiments, the spray container comprises an amount of the sprayable liquid composition sufficient for a single administration of the composition. In some embodiments, the spray container comprises an amount of the sprayable liquid composition sufficient for two administrations of the composition. In some embodiments, the spray container comprises an amount of the sprayable liquid composition sufficient for three or more administrations of the composition.

In some embodiments, the invention comprises a device that compartmentalizes the ingredients until time of application. For example, in one embodiment, one or more ingredients are separated from other ingredients until a user combines them. Such separate delivery helps to ensure that the two fluids are not mixed prematurely, and to ensure that minimal mixed composition is stored within the dispensing device, to ensure high efficacy. In some embodiments, a dual chamber or multi-chamber device is used to hold and/or apply the sprayable liquid composition. In some embodiments, the present disclosure provides a dual chamber device used to apply the compositions described herein. In some embodiments, the dual device chamber comprises a first and second chamber. In some embodiments, the first and second chamber each comprise different compositions. In some embodiments, the compositions in each chamber are kept separate until mixing during application either by concomitant actuation of the sprays from each of the chambers or by application from a pad when a seal is broken between pouches containing each formulation. Separate dip tubes may be provided to pull liquid from the separate chambers, each with its own separate pump. Each pump may deliver the separate compositions to a nozzle (e.g., a spray nozzle) of the device through separate delivery tubes (e.g., from the pumps to the nozzle). Such separate delivery tubes may be preferred for limiting mixing of the components before use. In another embodiment, a single delivery tube from the two pumps may be provided, e.g., where the volume of mixed composition that may be present within such a delivery tube may be sufficiently small so as to still provide overall desired efficacy characteristics.

In some embodiments, the first chamber comprises a first composition comprising 1% w/w to 10% w/w spironolactone, about 15% w/w to 20% w/w ethyl acetate and about 10% di-isopropyl adipate. In some embodiments, the second chamber comprises a second composition comprising about 50% w/w to about 60% w/w dimethyl Isosorbide, about 4% to 7% isopropyl myristate and about 1.5 w/w polyethylene glycol 400. In some embodiments, the dual chamber device comprises a first chamber comprising a first composition comprising 1% w/w to 10% w/w spironolactone, about 15% w/w to 20% w/w ethyl acetate and about 10% di-isopropyl adipate, and a second chamber comprising a second composition comprising about 50% w/w to about 60% w/w dimethyl Isosorbide, about 4% to 7% isopropyl myristate, and about 1.5 w/w polyethylene glycol 400, wherein the dual chamber device is suitable for providing a spray comprising the composition. In some embodiments, the first composition further comprises polyvinylpyrrolidone. In some embodiments, the contents of the first and second chambers are mixed during spraying of the composition onto human skin.

Particular embodiments of the present disclosure may include the administration of one or more additional active agents with the administration of minoxidil and, spironolactone and/or canrenone as described herein. The identity of the additional active agent(s) will be largely dependent on the nature of the underlying condition being treated (e.g., the addition of a moisturizer such as niacinamide may be appropriate in the treatment of skin dryness and the addition of agents such as finasteride and dutasteride may be appropriate in the treatment of hair loss in males and females. In some embodiments, the compositions of the device may include additional active agents that are effective in promoting hair growth or preventing hair loss in the treatment of male pattern baldness and/or ameliorating dry skin, rashes, facial flushing, raised red bumps, skin and skin sensitivity in the treatment of skin disorders.

Suitable additional active agents include, but are not limited to, minoxidil sulfate, or another salt form such as chloride, carbonate, nitrate, etc., finasteride, and niacinamide. In some embodiments, the additional active agent is finasteride, dutasteride, niacinamide or combinations thereof.

In some embodiments, the additional active agent is about 0.1% to about 10% of the liquid composition. In some embodiments, the additional active agent is about 1% w/w to about 10% w/w minoxidil, about 0.1% w/w to about 1.0% w/w finasteride or dutasteride, about 2% w/v to about 10% w/v niacinamide, or any combination thereof. In some embodiments, additional active agent is present in the same sprayable liquid composition as the spironolactone and/or minoxidil. For example, the additional active agent, e.g., minoxidil can be in the same composition as spironolactone, and the composition can be the same chamber of the dispensing system. In some embodiments, the additional active agent is in a sprayable liquid composition that is distinct from the sprayable liquid composition comprising the spironolactone. For example, the additional active agent, e.g., minoxidil, can be in a first composition in first chamber of a dispensing system, and the spironolactone can be in a second composition in a second chamber in the dispensing system. Thus, it is envisioned by the present disclosure that the both the spironolactone and the additional active agent can be applied from the same dispensing system independently of whether the spironolactone and the additional active agent are in the same sprayable liquid composition or in distinct sprayable liquid compositions.

In some embodiments, oral administration of spironolactone to a population of subjects have been reported to result in at least one adverse effect in a statistically measurable sub-population of subjects. Reported adverse effects and their incidence rates from clinical studies can include: (i) menstrual irregularities (oligomenorrhea, amenorrhea, breakthrough bleeding), (ii) 15-30% of women; breast tenderness or mastalgia: 5-15%; (iii) Dizziness or hypotension: 5-10%, (iv) Gastrointestinal upset (nausea, abdominal cramping): 3-8%, (v) Hyperkalemia: rare in healthy young women (<1%), but monitoring is still recommended, especially if other risk factors are present. See, e.g., Parisi et al., Dermatology Practical & Conceptual, 9(4), 321-327 (2019), Smith & van Neste, Journal of the American Academy of Dermatology, 84(2), 445-452 (2021), and Lee et al., International Journal of Women's Dermatology, 8(1), 15-22 (2022), each of which are incorporated by reference herein in its entirety.

Various containers can be used to hold, store or house the compositions disclosed herein in the spray container or dual chamber device. In some embodiments, the container or device can comprise a metal body, preferably lined with a chemically inert coating material to avoid degradation of the composition due to any interaction between the body and the composition. In some embodiments, a container or device can comprise a plastic body, preferably lined with a chemically inert coating material to avoid degradation of the composition due to any interaction between the body and the composition. In some embodiments, the container or device is a substantially rigid metal or plastic container adapted to contain a pressurized propellant located within the container and in contact with the product to be dispensed. In some embodiments, the container or device is an inner substantially rigid metal or plastic container adapted to contain a pressurized propellant located within an outer container made from the same material and away from contact with the product to be dispensed.

In some embodiments, the container or device body can be constructed from materials such as metal, glass, ceramics, polyester, polyethylene terephthalate (PET) or other polymers. In some embodiments, glass containers can be provided with a safety coating of, for instance, polypropylene to contain glass shards that may be formed on impact with a hard surface. In some embodiments, metal container bodies can be used to withstand impact and are amenable to surface coating. In some embodiments, the container comprises stainless steel, tinplate and aluminum, or combinations thereof. In some embodiments the aluminum is aluminum alloy or anodized aluminum.

Various inert coating materials can be used to line the container body including any suitable coating material known in the art such as a polymer, lacquer, resin or other coating treatment that creates a barrier between the container or device and the composition for preventing any chemical interaction between the composition and the container or device. In some embodiments, the inert material is a non-metallic coating. In some embodiments, known coatings for metal containers include acrylic, phenolic, polyester, epoxy and vinyl resins can be used to line the container body of the drug-device combination disclosed herein. Accordingly, the container or device coating for use with a composition of the present invention can be selected so that it exhibits no acidic or alkaline reactivity in itself, and that no acidic or alkaline reacting impurities are leached from it in the presence of the composition.

In some embodiments, the interior of a metal container or device can be lined with materials such as polyamides, polyimides, polypropylene, polyethylene, fluoropolymers, including perfluoroethylenepropylene copolymer (FEP), fluororubber (FPM), ethylene-propylene diene monomer rubber (EPDM), polytetrafluoroethylene (PTFE), ethylene tetrafluoroethylene copolymer (EFTE), perfluoroalkoxyalkanes, perfluoroalkoxyalkylenes, or blends of fluoropolymers with non-fluorocarbon polymers. Fluoropolymers can, for example, be used in combination with polyimide-polyamide resins.

The coating material of the container or device can be applied as a single layer, or in multiple layers, for example allowing each layer to cure before application of a further layer. The application of more than one coating can be used to shield the composition from the metal container or device and prevent adhesion of the active ingredients on the container or device walls.

The term “topical composition” in some embodiments can include the sprayable liquid composition on a surface, e.g., a skin surface, which forms when the aerosol spray comes in contact with the surface. In some embodiments, administration by aerosol spray provides a more consistent administration of the composition on the subject being treated. In some embodiments, administration by aerosol spray provides a more thorough administration of the composition to the subject, e.g., more area is covered. In some embodiments, administration by aerosol spray provides a more convenient method of administration. In some embodiments, administration by aerosol spray reduces the need for other applicators, e.g., wipes, gauzes, clothes, etc. In some embodiments, administration by aerosol spray reduces exposure of the hands or other unintended body parts to the presence of spironolactone and/or minoxidil that may occur by administration by creams, gels, or liquids. In some embodiments, administration by aerosol spray provides a more convenient mode of administration, as it allows the composition to dry quickly after application.

In embodiments in which the spray container is a pressurized device, the pressure in the sealed and pressurized device can be any pressure suitable for delivery of the sprayable liquid composition. In some embodiments, the pressure is about 28 psi to about 145 psi at 25 °C.

In some embodiments, the spray container comprising the sprayable liquid composition is a misting device, with a means for providing a mist of the topical composition. As used herein, the misting device would include any device which is capable of producing fine mist particles of the sprayable liquid composition. In some embodiments, the misting device produces mist particles of the sprayable liquid composition with an average diameter of about 20 μm to about 150 μm.

### Methods of Treatment

In some embodiments, the disclosure provides a method of treating male and female pattern hair loss or symptoms of polycystic ovary syndrome (PCOS), or combinations thereof in a subject, the method comprising topically applying any of the liquid compositions described herein as to a scalp surface of the subject.

The sprayable liquid compositions described herein can be used in the treatment of male and female pattern hair loss, hirsutism, hidradenitis suppurativa, polycystic ovary syndrome (PCOS), or combinations thereof in a subject. In some embodiments, the liquid compositions described herein can be used in the treatment of polycystic ovary syndrome (PCOS). In some embodiments, the subject is a human. In some embodiments, the human is a female. In some embodiments, the human is a male. When administered orally, spironolactone can cause adverse effects in males and females. The present disclosure provides a liquid composition of spironolactone and/or minoxidil that can provide fewer adverse events and can be well suited for both female and male subjects who currently have fewer options regarding the management of their male and female pattern hair loss.

The term “treatment” as used herein refers to reducing the severity of a symptom associated with male and female pattern hair loss and PCOS, or combinations thereof. Thus, in some embodiments, methods provided herein provide palliative care of male and female pattern hair loss

The term “subsequent” as used herein refers to applications of the liquid compositions that follow or come after another application in time, order, or place. For example, an application of the liquid composition that is 24 hours after the first application of the composition to the skin is a subsequent application.

In some embodiments, the subsequent application is applied one hour to one week after the first application. In some embodiments, the subsequent application is applied two hours to 4 days after the first application. In some embodiments, the subsequent application is applied four hours to two days after the first application. In some embodiments, the subsequent application is applied six hours to one day after the first application. In some embodiments, the subsequent application is applied twelve hours to one day after the first application. In some embodiments, the subsequent application is applied “as needed” or upon the occurrence of a symptom associated with acne, male and female pattern hair loss

In some embodiments, the first application and the second application are applied at the same site on the skin surface of the subject. In some embodiments, the second application of the composition is applied at a site on the skin that is the site of the first application.

In some embodiments, the first application of the composition is applied at a site on the skin that is different from the second site. In some embodiments, the first application and the second application are applied at different sites on the skin surface of the subject.

The sprayable liquid compositions described herein can be applied in a volume of about 1 mL or less in a single actuation. In some embodiments, the composition is applied in a volume of about 750 mL or less in a single actuation. In some embodiments, the composition is applied in a volume of about 1 mL or less in a single actuation. In some embodiments, the composition is applied in a volume of about 500 μL or less in a single actuation. In some embodiments, the composition is applied in a volume of about 250 μL or less in a single actuation.

In some embodiments, the present disclosure provides for liquid compositions that can be applied using a dropper that dispenses solutions.

In some embodiments, the present disclosure provides for sprayable liquid compositions that require a reduced frequency of administrations for treatment of conditions described herein. For example, in some embodiments, the composition is administered not more than once in a 24-hour period to alleviate symptoms of the conditions described herein.

All references cited herein, including patents, patent applications, papers, textbooks and the like, and the references cited therein, to the extent that they are not already, are hereby incorporated herein by reference in their entirety.

### EXAMPLES

### Example 1—Developing Sprayable Liquid Compositions of Spironolactone

Compositions 1-28 in Tables 1~4 comprising minoxidil and, spironolactone and/or canrenone are prepared according to the manufacturing process described herein. To prepare the compositions, di-isopropyl adipate, dimethyl isosorbide, dimethyl sulphoxide, ethyl acetate, are mixed in a suitable vessel to produce a solvent mixture. The penetration enhancers, 1-dodecylazacycloheptan-2-one, isopropyl myristate, octisalate, oleic acid, or diethylene glycol monoethyl ether (Transcutol® P), and/or a fragrance are then added to the mixture and the mixture is further stirred. The active ingredients minoxidil and, spironolactone and/or canrenone are then added to the mixture and the mixture again stirred until a clear solution is achieved. Cetearyl alcohol and polythene glycol are added and stirred until a translucent solution is formed. Lastly, the solution is added to a suitable container and a spray pump is affixed to the container. Exemplary minoxidil and, spironolactone and/or canrenone compositions are shown below.

TABLE 1

| | Composition | | | | | | |
|---|---|---|---|---|---|---|---|
| | 1 | 2 | 3 | 4 | 5 | 6 | 7 |
| Ingredient | % w/w | | | | | | |
| Spironolactone | 1.0 | 2.0 | 3.0 | 4.0 | 5.0 | 8.0 | 10.0 |
| Minoxidil | 1.0 | 2.0 | 3.0 | 4.0 | 5.0 | 8.0 | 10.0 |
| Ethyl Acetate | 30.0 | 41.5. | 47.9 | 51.0 | 40.0 | 35.0 | 29.6 |
| Di-isopropyl adipate | 30.0 | 14.5 | 12.4 | 10.5 | 10.0 | 10.0 | 10.5 |
| Dimethyl Isosorbide | 10.0 | 10.0 | 13.5 | 10.0 | 10.0 | 7.5 | 10.0 |
| Dimethyl sulfoxide | 5.0 | 5.0 | 5.0 | 5.0 | 5.0 | 5.0 | 5.0 |
| Glycerol | 5.0 | 5.0 | 5.0 | 5.0 | 5.0 | 5.0 | 5.0 |
| Iso-propyl Myristate | 6.0 | 6.0 | 6.0 | 6.0 | 6.0 | 6.0 | 6.0 |
| Oleic Acid | 4.0 | 6.0 | 7.0 | 5.0 | 6.0 | 7.0 | 6.0 |
| Cetearyl Alcohol | 5.5 | 5.0 | 7.0 | 7.0 | 5.0 | 7.0 | 5.0 |
| Polyethylene | 1.5 | 1.0 | 1.2 | 1.5 | 1.0 | 1.5 | 0.9 |
| glycol 400 | | | | | | | |
| Fragrance | 0.5 | 0.5 | 0.5 | 0.5 | 0.5 | 0.5 | 0.5 |
| Preservative | 1.0 | 1.0 | 1.0 | 1.0 | 1.0 | 1.0 | 1.0 |
| Antioxidant | 0.5 | 0.5 | 0.5 | 0.5 | 0.5 | 0.5 | 0.5 |
| TOTAL | 100 | 100.0 | 100.0 | 100.0 | 100.0 | 100.0 | 100.0 |

TABLE 2

| | Composition | | | | | | |
|---|---|---|---|---|---|---|---|
| | 8 | 9 | 10 | 11 | 12 | 13 | 14 |
| Ingredient | % w/w | | | | | | |
| Spironolactone | 2.0 | 4.0 | 5.0 | 6.0 | 8.0 | 10.0 | 15.0 |
| Minoxidil | 2.0 | 4.0 | 5.0 | 6.0 | 8.0 | 10.0 | 15.0 |
| Oleic Acid | 7.0 | 7.0 | 7.0 | 7.0 | 7.0 | 7.0 | 7.0 |
| Isopropyl Myristate | 15.0 | 15.0 | 15.0 | 15.0 | 14.5 | 15.0 | 15.0 |
| Glycerol | 8.0 | 8.0 | 8.0 | 8.0 | 8.0 | 8.0 | 4.5 |
| Cetearyl Alcohol | 2.0 | 2.0 | 2.0 | 2.0 | 2.0 | 2.0 | 1.0 |
| Dimethyl- | 20.0 | 15.0 | 13.0 | 13.0 | 15.0 | 13.0 | 15.0 |
| isosorbide | | | | | | | |
| Transcutol ® P | 15.0 | 15.0 | 15.0 | 15.0 | 15.0 | 15.0 | 15.0 |
| Polyethylene | 26.5 | 30..0 | 27.5 | 25.5 | 20.0 | 27.5 | 10.0 |
| Glycol 400 | | | | | | | |
| Phenoxyethanol | 1.0 | 1.0 | 1.0 | 1.0 | 1.0 | 1.0 | 1.0 |
| Alpha Tocopherol | 1.0 | 1.0 | 1.0 | 1.0 | 1.0 | 1.0 | 1.0 |
| Fragrance | 0.5 | 0.5 | 0.5 | 0.5 | 0.5 | 0.5 | 0.5 |
| TOTAL | 100.0 | 100.0 | 100.0 | 100.0 | 100.0 | 100.0 | 100.0 |

TABLE 3

| | Composition | | | | | | |
|---|---|---|---|---|---|---|---|
| | 15 | 16 | 17 | 18 | 19 | 20 | 21 |
| Ingredient | % w/w | | | | | | |
| Spironolactone USP | 1.0 | 2.0 | 4.0 | 6.0 | 8.0 | 10.0 | 20.0 |
| Minoxidil USP | 1.0 | 2.0 | 2.0 | 6.0 | 8.0 | 10.0 | 20.0 |
| Ethyl Acetate USP | 15.0 | 18.0 | 15.0 | 13.5 | 13.5 | 10.0 | 5.0 |
| Di-isopropyl | 15.0 | 16.5 | 11.4 | 10.0 | 15.0 | 10.0 | 5.0 |
| adipate USP | | | | | | | |
| Dimethyl | 15.0 | 15.0 | 15.0 | 15.0 | 15.0 | 15.0 | 15.0 |
| Isosorbide USP | | | | | | | |
| Dimethyl | 5.0 | 5.0 | 5.0 | 5.0 | 5.0 | 0.0 | 0.0 |
| Sulphoxide USP | | | | | | | |
| Oleic Acid USP | 13.5 | 15.0 | 15.0 | 15.0 | 15.0 | 15.0 | 6.5 |
| Glycerol | 7.0 | 4.0 | 7.0 | 7.0 | 5.0 | 7.0 | 7.0 |
| Polyethylene | 25.0 | 20.0 | 22.1 | 20.0 | 20.0 | 18.5 | 20.0 |
| glycol 400 | | | | | | | |
| Sodium Lauryl | 0.5 | 0.5 | 0.5 | 0.5 | 0.5 | 0.5 | 0.5 |
| Sulphate | | | | | | | |
| Phenoxyethanol | 1.0 | 1.0 | 1.0 | 1.0 | 1.0 | 1.0 | 1.0 |
| Alpha Tocopherol | 0.5 | 0.5 | 0.5 | 0.5 | 0.5 | 0.5 | 0.5 |
| Fragrance | 0.5 | 0.5 | 0.5 | 0.5 | 0.0 | 0.5 | 0.5 |
| TOTAL | 100.0 | 100.0 | 100.0 | 100.0 | 100.0 | 100.0 | 100.0 |

### Example 2—Dual Chamber Spironolactone Spray Compositions

Exemplary compositions for a dual chamber dispensing systems are outlined in Table 4 below. Solutions from both chambers are kept separate until mixing during application either by concomitant actuation of the sprays from each of the chambers or by application from a pad when a seal is broken between pouches containing each formulation.

TABLE 4

| | Composition | | | | | | |
|---|---|---|---|---|---|---|---|
| | 22 | 23 | 24 | 25 | 26 | 27 | 28 |
| Ingredients (Chamber 1) | % w/w | | | | | | |
| Spironolactone USP | 5.0 | 5.0 | 5.0 | 5.0 | 5.0 | 5.0 | 10.0 |
| Ethylacetate USP | 19.0 | 19.0 | 19.0 | 15.0 | 20.0 | 15.0 | 15.0 |
| Di-isopropyl adipate USP | 10.0 | 10.0 | 10.0 | 10.0 | 10.0 | 10.0 | 10.0 |
| Ingredients (Chamber 2) | % w/w | | | | | | |
| Minoxidil | 5.0 | 5.0 | 5.0 | 5.0 | 5.0 | 5.0 | 10.0 |
| Dimethyl Isosorbide USP | 45.0 | 45.0 | 42.0 | 48.0 | 44.0 | 46.0 | 38.5 |
| Transcutol P | 4.0 | 6.0 | 7.0 | 5.0 | 6.0 | 7.0 | 6.0 |
| Isopropyl Myristate USP | 7.0 | 5.0 | 7.0 | 7.0 | 5.0 | 7.0 | 5.5 |
| Polyethylene Glycol 400 | 5.0 | 5.0 | 5.0 | 5.0 | 5.0 | 5.0 | 5.0 |
| TOTAL | 100.0 | 100.0 | 100.0 | 100.0 | 100.0 | 100.0 | 100.0 |

1. A liquid composition comprising:
a. about 1% w/v to about 20% w/v spironolactone, canrenone or a combination of both;
b about 1% w/v to about 20% w/v minoxidil;
c. about 10% w/v to about 60% w/v C₄-C₁₄ aliphatic solvent;
d. a penetration enhancer;
e. about 1% w/v to about 30% w/v polyethylene glycol
wherein the composition does not comprise C₁-C₃ alcohols.

2. (canceled)

3. The composition of claim 1, wherein the composition comprises about 5% w/v spironolactone and about 5% w/v of minoxidil.

4. The composition of claim 1, wherein the aliphatic solvent is di-isopropyl adipate, dimethyl isosorbide, dimethyl sulphoxide, ethyl acetate, dimethyl isosorbide, or combinations thereof.

5. The composition of claim 1, wherein the aliphatic solvent is
a. about 15% w/v to about 60% w/v ethyl acetate;
b. about 10% w/v to about 65% w/v di-isopropyl adipate; and
c. about 5% w/v to about 60% w/v dimethyl isosorbide.

6. The composition of claim 1, wherein the penetration enhancer is azone, isopropyl myristate, octisalate, oleic acid, Transcutol® P or combinations thereof.

7. The composition of claim 1, wherein the penetration enhancer is about 1% to about 30% w/v of the composition.

8. (canceled)

9. (canceled)

10. The composition of claim 1, wherein the polyethylene glycol is polyethylene glycol 400.

11. The composition of claim 1, wherein the composition further comprises a buffer.

12. (canceled)

13. The composition of claim 1, wherein the composition further comprises an emollient, humectant and a conditioner.

14. (canceled)

15. (canceled)

16. The composition of claim 1, wherein the composition further comprises an additional active agent.

17. (canceled)

18. The composition of claim 1, wherein the oxygen (O₂) content in the composition is less than 2%.

19. (canceled)

20. The composition of claim 16, wherein the additional active agent is
a. about 0.1% w/w to about 1.0% w/w finasteride;
b. about 0.1% w/w to about 1.0% w/w dutasteride; or
c. about 2% w/v to about 10% w/v niacinamide.

21. The composition of claim 1, wherein the pH of the composition is about 4 to about 6.

22. (canceled)

23. The composition of claim 1, wherein the composition comprises an antioxidant, a preservative and/or a fragrance.

24. The composition of claim 1, wherein the composition has a viscosity of 100-2000 cPs at room temperature.

25. The composition of claim 1, wherein the composition does not comprise ethanol, propanol, isopropanol, or propylene glycol.

26. (canceled)

27. The composition of claim 1, wherein the composition is in the form of a solution, cream, gel, foam or a spray.

28. A method of treating hair loss in a subject, the method comprising topically applying the composition of claim 1 to a scalp surface of the subject.

29-35. (canceled)

36. The method of claim 28, wherein the composition is applied to the scalp in a single actuation in a volume of about 1 mL or less using a spray container comprising:
a. the composition; and
b. a metering valve.

37-39. (canceled)

40. A dual chamber device comprising:
a. a first chamber comprising a first composition comprising 1% w/w to 10% w/w spironolactone, about 15% w/w to 20% w/w ethyl acetate and about 10% di-isopropyl adipate.
b. a second chamber comprising a second composition comprising 1% w/w to 10% w/w of minoxidil and comprising about 40% w/w to about 50% w/w dimethyl isosorbide, about 4% to 7% Transcutol® P, about 5% w/v to about 7% w/v isopropyl myristate and about 5.0 w/w polyethylene glycol 400,
wherein the dual chamber device is suitable for providing a spray comprising the composition.

41-43. (canceled)
